(19) United States
(12) Patent Application Publication
(10) Pub. No.: US 2025/0381171 A1
(43) Pub. Date: Dec. 18, 2025

(54) COMBINATION OF DIHYDROBERBERINE AND ERGOTHIONEINE TO REDUCE OR INHIBIT ADVANCED GLYCATION END-PRODUCTS

(71) Applicant: NANJING NUTRABUILDING BIO-TECH CO., LTD., Nanjing (CN)

(72) Inventors: Ronghua YI, Nanjing (CN); Kylin LIAO, Nanjing (CN); Shawn WELLS, Frisco, TX (US)

(21) Appl. No.: 19/314,867

(22) Filed: Aug. 29, 2025

Related U.S. Application Data
(63) Continuation of application No. PCT/CN2024/078677, filed on Feb. 27, 2024.

(30) Foreign Application Priority Data
Mar. 1, 2023 (WO) PCT/CN2023/079077

(51) Int. Cl. A61K 31/4375; A61K 8/49; A61K 31/4172; A61P 3/10; A61P 39/06; A61Q 19/08

(52) U.S. Cl. CPC A61K 31/4375; A61K 8/4946; A61K 31/4172; A61P 3/10; A61P 39/06; A61Q 19/08

(57) ABSTRACT

The present invention provides novel methods for reducing or inhibiting advanced glycation end products (AGEs) in a subject, comprising administration to the subject a composition comprising an effective amount of dihydroberberine or a pharmaceutically acceptable salt, acid, ester, analog or derivative thereof; and an effective amount of ergothioneine or a pharmaceutically acceptable salt, acid, ester, analog or derivative thereof. The present invention also provides a composition comprising an effective amount of dihydroberberine or a pharmaceutically acceptable salt, acid, ester, analog or derivative thereof; and an effective amount of ergothioneine or a pharmaceutically acceptable salt, acid, ester, analog or derivative thereof, for reducing or inhibiting advanced glycation end products (AGEs) in a subject.

### CROSS REFERENCE TO RELATED APPLICATION

This application is a continuation application of International Patent Application No. PCT/CN2024/078677, filed on Feb. 27, 2024, which claims the priority of the International Patent Application No. PCT/CN2023/079077, filed on Mar. 1, 2023, the contents of all of which are incorporated herein by reference in their entirety.

### BACKGROUND OF THE INVENTION

### Technical Field

The present invention relates to compositions and methods for reducing or inhibiting advanced glycation end products (AGEs), especially for anti-glycation, or mitigating or preventing aging in a subject. The composition may comprise an effective amount of dihydroberberine or a pharmaceutically acceptable salt, acid, ester, analog or derivative thereof; and an effective amount of ergothioneine or a pharmaceutically acceptable salt, acid, ester, analog or derivative thereof.

### Background Technology

Advanced glycation end-products (AGEs) are modifications of proteins or lipids that become nonenzymatically glycated and oxidized after contact with aldose sugars. Early glycation and oxidation processes result in the formation of Schiff bases and Amadori products. Further glycation of proteins and lipids causes molecular rearrangements that lead to the generation of AGEs. AGEs may fluoresce, produce reactive oxygen species (ROS), bind to specific cell surface receptors, and form cross-links. Alternatively, they can undergo further oxidation, dehydration, polymerization and oxidative breakdown reactions to give rise to numerous other AGEs. Oxygen, reactive oxygen species (ROS) and redox active transition metals accelerate AGE formation. When an oxidative step is involved, the products are called AGEs. The gradual accumulation of AGEs in vivo in hyperglycemic environments and during aging will cause many bad effects, like aging.

AGEs could occur by various exogenous and endogenous reasons. With respect to exogenous reasons, UV irradiation is one of most crucial exogenous reasons, which could induce oxidative stress and lipid peroxidase-like malondialdehyde (MDA) and the activation of matrix metalloproteinase (MMP). Moreover, many literature indicated that ultraviolet (UV) irradiation may precipitate the formation of AGEs in vivo. Another one is a higher dietary intake of carbohydrates, inducing high glucose levels The known mechanisms by which carbohydrates result in oxidative stress are the activation of mitochondrial oxidative metabolism of glucose, which leads to the generation of ROS. Regarding to endogenous reasons, the primary cause is the accumulation of oxidative damage caused by ROS.

Up to now, there are some synthetic inhibitors to alleviate the development of AGEs, such as calcium antagonists, amlodipine, kinetin, quinine, etc. However, there is a safety concerns and side effects of these synthetic inhibitors, like weakened liver, anemia, vomiting, gastrointestinal disorders, diarrhea, dizziness, headache flu. Thus, it is understood that, the importance and urgency of having available alternatives which reduce or even inhibit the development of AGEs may be of great attention.

Dihydroberberine (DHB) could be beneficial in alleviating the development of AGEs by scavenging chronic ROS, reducing the level of MDA and the expression of MMP-1. Moreover, we found another efficient ingredient, ergothioneine (EGT, C₉H₁₅N₃O₂S), a powerful antioxidant. It reported that EGT could protect against oxidative damage induced by UV. Also, EGT was useful to the body for combating oxidative stress (OS), a cellular imbalance that may result from poor diets or unhealthy lifestyles but also potentially as a consequence of the ageing process. In addition, EGT is concentrated in cells and tissues wherein exposed to OS frequently, such as ocular tissues, liver, bone marrow and seminal fluid, etc. Hence, EGT could scavenge chronic ROS, to prevent the process of AGEs.

Taken together, in this invention, we found that DHB or a pharmaceutically acceptable salt, acid, ester, analog or derivative thereof, EGT or a pharmaceutically acceptable salt, acid, ester, analog or derivative thereof, and especially the combination of them could be considered as a promising and attractive ingredient to attenuate the process of AGEs, thus inhibiting glycation, mitigating or preventing aging.

### SUMMARY OF THE PRESENT INVENTION

In a first aspect, the present invention provides a method for reducing or inhibiting advanced glycation end products (AGEs) in a subject, comprising administrating to the subject a composition comprising: an effective amount of dihydroberberine or a pharmaceutically acceptable salt, acid, ester, analog or derivative thereof; and an effective amount of ergothioneine or a pharmaceutically acceptable salt, acid, ester, analog or derivative thereof.

In some embodiments, the method is used for anti-glycation.

In some embodiments, the method is used for mitigating or preventing aging.

In some embodiments, the aging is characterized by fine wrinkles, loss of elasticity, orange-peel or dull appearance of skin, reduced epidermal and dermal thickness, epidermal atrophy, decreased mitotic rate of basal keratinocytes, decreased proliferative capacity, cellular senescence, atrophy of dermal extracellular matrix, change of physiological properties of connective tissues.

In some embodiments, the reducing or inhibiting advanced glycation end products (AGEs) is achieved by controlling blood glucose, scavenging chronic ROS, reducing H₂O₂ and MDA productions and/or expression of MMP-1.

In some embodiments, the scavenging chronic ROS includes improving activities of anti-oxidative enzymes, such as SOD, CAT, GSH, and GSH-Px.

In some embodiments, the dihydroberberine or a pharmaceutically acceptable salt, acid, ester, analog or derivative thereof is administrated at a daily dose of 1-2000 mg, the ergothioneine or a pharmaceutically acceptable salt, acid, ester, analog or derivative thereof is administrated at a daily dose of 1-1500 mg. In some embodiments, the dihydroberberine or a pharmaceutically acceptable salt, acid, ester, analog or derivative thereof may be administrated at a daily dose of 2-1500 mg, 5-1000 mg, 10-800 mg, 20-600 mg, 50-500 mg, 60-500 mg, 80-400 mg, or 100-400 mg, the ergothioneine or a pharmaceutically acceptable salt, acid, ester, analog or derivative thereof may be administrated at a daily dose of 2-1000 mg, 2-500 mg, 3-100 mg, 3-50 mg, or 5-50 mg. In some embodiments, the daily dose is administered in divided doses or a single dose. In some embodiments, the administration is at least once a day or more times a day. In some embodiments, the administration is at least 7 days and above in one period.

In some embodiments, the ratio of the dihydroberberine or a pharmaceutically acceptable salt, acid, ester, analog or derivative thereof and the ergothioneine or a pharmaceutically acceptable salt, acid, ester, analog or derivative thereof ranges from 1:200 to 800:1. In some embodiments, the ratio of the dihydroberberine or a pharmaceutically acceptable salt, acid, ester, analog or derivative thereof and the ergothioneine or a pharmaceutically acceptable salt, acid, ester, analog or derivative thereof may also be 1:100 to 400:1, 1:100 to 200:1, 1:50 to 200:1, 1:50 to 100:1, 1:25 to 100:1, 1:20 to 100:1, 1:20 to 80:1, 1:15 to 80:1, 1:10 to 80:1, 1:8 to 80:1, 1:8 to 60:1, 1:5 to 60:1, 1:5 to 40:1, 1:3 to 40:1, 1:3 to 30:1, 1:2 to 20:1, 1:2 to 10:1, 1:1 to 10:1, 1:1 to 5:1, or 1:1.

In some embodiments, the subject is human or cattle or pet. In some embodiments, the subject is human.

In some embodiments, the composition is prepared as a food, a drink, a supplement, a cosmetic product, or a pharmaceutical formulation.

In some embodiments, the administration is through various routes selected from oral administration, intravenous injection, intramuscular injection, intraperitoneal injection, topical application, or sublingual application.

In some embodiments, the composition is formulated in solutions, liquid suspensions, parenteral solutions, injections, tablets, pills, granules, powders, films, suppositories, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

In a second aspect, the present invention provides a composition comprising an effective amount of dihydroberberine or a pharmaceutically acceptable salt, acid, ester, analog or derivative thereof; and an effective amount of ergothioneine or a pharmaceutically acceptable salt, acid, ester, analog or derivative thereof, for reducing or inhibiting advanced glycation end products (AGEs) in a subject.

In some embodiments, the composition is used for anti-glycation.

In some embodiments, the composition is used for mitigating or preventing aging.

In some embodiments, the aging is characterized by fine wrinkles, loss of elasticity, orange-peel or dull appearance of skin, reduced epidermal and dermal thickness, epidermal atrophy, decreased mitotic rate of basal keratinocytes, decreased proliferative capacity, cellular senescence, atrophy of dermal extracellular matrix, change of physiological properties of connective tissues.

In some embodiments, the reducing or inhibiting advanced glycation end products (AGEs) is achieved by controlling blood glucose, scavenging chronic ROS, reducing H₂O₂ and MDA productions and/or expression of MMP-1.

In some embodiments, the scavenging chronic ROS includes improving activities of anti-oxidative enzymes, such as SOD, CAT, GSH, and GSH-Px.

In some embodiments, the dihydroberberine or a pharmaceutically acceptable salt, acid, ester, analog or derivative thereof is administrated at a daily dose of 1-2000 mg, the ergothioneine or a pharmaceutically acceptable salt, acid, ester, analog or derivative thereof is administrated at a daily dose of 1-1500 mg. In some embodiments, the dihydroberberine or a pharmaceutically acceptable salt, acid, ester, analog or derivative thereof may be administrated at a daily dose of 2-1500 mg, 5-1000 mg, 10-800 mg, 20-600 mg, 50-500 mg, 60-500 mg, 80-400 mg, or 100-400 mg, the ergothioneine or a pharmaceutically acceptable salt, acid, ester, analog or derivative thereof may be administrated at a daily dose of 2-1000 mg, 2-500 mg, 3-100 mg, 3-50 mg, or 5-50 mg. In some embodiments, the daily dose is administered in divided doses or a single dose. In some embodiments, the administration is at least once a day or more times a day. In some embodiments, the administration is at least 7 days and above in one period.

In some embodiments, the ratio of the dihydroberberine or a pharmaceutically acceptable salt, acid, ester, analog or derivative thereof and the ergothioneine or a pharmaceutically acceptable salt, acid, ester, analog or derivative thereof ranges from 1:200 to 800:1. In some embodiments, the ratio of the dihydroberberine or a pharmaceutically acceptable salt, acid, ester, analog or derivative thereof and the ergothioneine or a pharmaceutically acceptable salt, acid, ester, analog or derivative thereof may also be 1:100 to 400:1, 1:100 to 200:1, 1:50 to 200:1, 1:50 to 100:1, 1:25 to 100:1, 1:20 to 100:1, 1:20 to 80:1, 1:15 to 80:1, 1:10 to 80:1, 1:8 to 80:1, 1:8 to 60:1, 1:5 to 60:1, 1:5 to 40:1, 1:3 to 40:1, 1:3 to 30:1, 1:2 to 20:1, 1:2 to 10:1, 1:1 to 10:1, 1:1 to 5:1, or 1:1.

In some embodiments, the subject is human or cattle or pet. In some embodiments, the subject is human.

In some embodiments, the composition is prepared as a food, a drink, a supplement, a cosmetic product, or a pharmaceutical formulation.

In some embodiments, the composition is formulated in solutions, liquid suspensions, parenteral solutions, injections, tablets, pills, granules, powders, films, suppositories, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

In a third aspect, the present invention provides use of a composition for preparing food, drink, supplement, cosmetic product, or pharmaceutical formulation for reducing or inhibiting advanced glycation end products (AGEs) in a subject, wherein the composition comprises an effective amount of dihydroberberine or a pharmaceutically acceptable salt, acid, ester, analog or derivative thereof; and an effective amount of ergothioneine or a pharmaceutically acceptable salt, acid, ester, analog or derivative thereof.

In some embodiments, the composition is used for anti-glycation.

In some embodiments, the composition is used for mitigating or preventing aging.

In some embodiments, the aging is characterized by fine wrinkles, loss of elasticity, orange-peel or dull appearance of skin, reduced epidermal and dermal thickness, epidermal atrophy, decreased mitotic rate of basal keratinocytes, decreased proliferative capacity, cellular senescence, atrophy of dermal extracellular matrix, change of physiological properties of connective tissues.

In some embodiments, the reducing or inhibiting advanced glycation end products (AGEs) is achieved by controlling blood glucose, scavenging chronic ROS, reducing H₂O₂ and MDA productions and/or expression of MMP-1.

In some embodiments, the scavenging chronic ROS includes improving activities of anti-oxidative enzymes, such as SOD, CAT, GSH, and GSH-Px.

In some embodiments, the dihydroberberine or a pharmaceutically acceptable salt, acid, ester, analog or derivative thereof is administrated at a daily dose of 1-2000 mg, the ergothioneine or a pharmaceutically acceptable salt, acid, ester, analog or derivative thereof is administrated at a daily dose of 1-1500 mg. In some embodiments, the dihydroberberine or a pharmaceutically acceptable salt, acid, ester, analog or derivative thereof may be administrated at a daily dose of 2-1500 mg, 5-1000 mg, 10-800 mg, 20-600 mg, 50-500 mg, 60-500 mg, 80-400 mg, or 100-400 mg, the ergothioneine or a pharmaceutically acceptable salt, acid, ester, analog or derivative thereof may be administrated at a daily dose of 2-1000 mg, 2-500 mg, 3-100 mg, 3-50 mg, or 5-50 mg. In some embodiments, the daily dose is administered in divided doses or a single dose. In some embodiments, the administration is at least once a day or more times a day. In some embodiments, the administration is at least 7 days and above in one period.

In some embodiments, the ratio of the dihydroberberine or a pharmaceutically acceptable salt, acid, ester, analog or derivative thereof and the ergothioneine or a pharmaceutically acceptable salt, acid, ester, analog or derivative thereof ranges from 1:200 to 800:1. In some embodiments, the ratio of the dihydroberberine or a pharmaceutically acceptable salt, acid, ester, analog or derivative thereof and the ergothioneine or a pharmaceutically acceptable salt, acid, ester, analog or derivative thereof may also be 1:100 to 400:1, 1:100 to 200:1, 1:50 to 200:1, 1:50 to 100:1, 1:25 to 100:1, 1:20 to 100:1, 1:20 to 80:1, 1:15 to 80:1, 1:10 to 80:1, 1:8 to 80:1, 1:8 to 60:1, 1:5 to 60:1, 1:5 to 40:1, 1:3 to 40:1, 1:3 to 30:1, 1:2 to 20:1, 1:2 to 10:1, 1:1 to 10:1, 1:1 to 5:1, or 1:1.

In some embodiments, the composition is formulated in solutions, liquid suspensions, parenteral solutions, injections, tablets, pills, granules, powders, films, suppositories, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

These and other features, aspects, and advantages of the present invention will become better understood with reference to the following description and appended claims.

### BRIEF DESCRIPTION OF THE DRAWINGS

FIG. 1 is a graph of a percentage inhibition of fluorescence of the AGEs relative to the control.

FIG. 2 is a graph of blood glucose levels of four groups of mice at the beginning of the trial (0 week), after the D-galactose injection (8th week), and at the end of the trial (14th week).

FIG. 3 is a graph of MDA content of four groups of mice at the end of the trial (14th week).

FIG. 4 is a graph of H₂O₂ content of four groups of mice at the end of the trial (14th week).

FIG. 5 is a graph of SOD activity of four groups of mice at the end of the trial (14th week).

FIG. 6 is a graph of CAT activity of four groups of mice at the end of the trial (14th week).

FIG. 7 is a graph of GSH activity of four groups of mice at the end of the trial (14th week).

FIG. 8 is a graph of GSH-Px activity of four groups of mice at the end of the trial (14th week).

FIG. 9 is a graph of MMP-1 content of four groups of mice at the end of the trial (14th week).

### DETAILED DESCRIPTION OF THE INVENTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation wherein said terms are used in their non-limiting sense to mean that items following the word are included, but items not specifically mentioned are not excluded. It also encompasses the more limiting verb ‘to consist essentially of’ and ‘to consist of’.

As used herein, the term “effective amount” refers to the amount required to achieve the effect as taught herein. The specific effective dose level for any particular subject will depend upon a variety of factors including the conditions being treated and the severity of the conditions; the specific composition employed; the age, body weight, general health, sex and diet of the subject; the time of administration, route of administration, and rate of excretion of dihydroberberine and/or ergothioneine or its analog or its derivatives employed; the duration of the treatment; and like factors well known in the medical arts. For example, it is well known within the skill of the art to start doses of the compound at levels lower than those required to achieve the desired effect and to gradually increase the dosage until the desired effect is achieved.

One of skill in the art recognizes that an amount may be considered “effective” even if the condition is not totally eradicated or prevented, but it or its symptoms and/or effects are improved or alleviated partially in the subject.

As used herein, the term “pharmaceutically acceptable” means pharmaceutically, physiologically, alimentarily, and/or nutritionally acceptable, and refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

As used herein, the term “mammal” or “subject” may be used interchangeably to refer to any animal to which the presently disclosed methods and compositions may be applied or administered. The animal may have an illness or other disease, but the animal does not need to be sick to benefit from the presently disclosed methods and compositions. As such any animal may apply the disclosed combinations, compositions or kits, or be a recipient of the disclosed methods. “Mammal” includes, without limitation, mice, rats, rabbits, guinea pigs, dogs, cats, sheep, goats, cows, horses, primates, such as monkeys, chimpanzees, and apes, and, in particular, humans. Although the animal subject is preferably a human, the methods and compositions of the invention have application in veterinary medicine.

The dosage of dihydroberberine and/or ergothioneine, or a pharmaceutically acceptable salt, acid, ester, polymer, analog or derivative thereof and/or composition comprising the same may range broadly, depending upon the desired effects and the indication. The daily dosage regimen for an adult human patient may be, for example, an oral dose of between 0.01 mg and 3000 mg of dihydroberberine and/or ergothioneine or its analog or derivative, preferably between 1 mg and 700 mg, e.g., 5 to 200 mg, or between about 0.1 mg and about 1,000 mg of dihydroberberine and/or ergothioneine or its analog or derivative per kg of body weight of the subject. The dosage may be a single one or a series of two or more given in the course of one or more days, as is needed by the subject. In some embodiments, the compounds are administered for a period of time, for example for a week or more, or for months or years.

As used herein, the term “administration” refers to the process of delivering a disclosed composition or active ingredient to a subject. The compositions of the invention can be administered in a variety of ways, including orally, intragastrically, and parenterally (e.g., intravenous and intraarterial as well as other suitable parenteral routes), and the like.

As used herein, a “parenteral solution” refers to a solution that can be administered elsewhere in the body than the mouth and alimentary canal. It is not delivered via the intestinal tract. For example, parenteral solution can be delivered intravenously.

As used herein, a “tonic” refers to a medicinal substance taken to give a feeling of vigor or well-being.

As used herein, a “syrup” refers to a thick sticky liquid derived from a sugar-rich plant, for example, sugar cane, corn, and maple.

Multiple techniques of administering a composition exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

“Intraperitoneal” as used here means within or administered through the peritoneum. The peritoneum is a thin, transparent membrane that lines the walls of the abdominal (peritoneal) cavity and contains/encloses the abdominal organs such as the stomach and intestines.

As used herein, “sublingual” refers to situated or applied under the tongue.

A “functionalized cream composition” includes a cream composition that has a potentially positive effect on health beyond basic nutrition.

An “essence” includes an extract or concentrate obtained from a particular plant or other matter and used for flavoring or scent. A “functionalized essence” includes an essence that has a potentially positive effect on health beyond basic nutrition.

A “functionalized food composition” includes a food composition that has a potentially positive effect on health beyond basic nutrition.

Any titles or subheadings used herein are for organization purposes and should not be used to limit the scope of embodiments disclosed herein.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

All publications and patent applications cited in this specification are herein incorporated by reference as if each individual publication or patent application were specifically and individually indicated to be incorporated by reference.

### EXAMPLES

### Example 1

### Measurement of Glycation

The formulation of glycation products was observed placing together a bovine serum albumin (BSA) and D-ribose in the absence of DHB and EGT, EGT only and DHB and EGT combination.

Specifically, a solution of BSA at a concentration of 1 mg/ml dissolved in phosphate-buffered saline (PBS) was incubated at 37° C. for 10 days with D-ribose at a concentration of 10 mM, in the presence of EGT only (3 mM, Group 1) and the DHB (3 mM) and EGT (3 mM) combination (Group 2).

As known, the final glycation end products exhibit light emission in the range of 420-600 nm after light excitation with a length of 300-420 nm. So, the glycation was evaluated by measuring the fluorescence of the AGEs at λem=450 nm emitted by each sample after excitation at λex=380 nm. The inhibition of glycation was visualized by the decrease in fluorescence compared with the samples treated with the sugar alone.

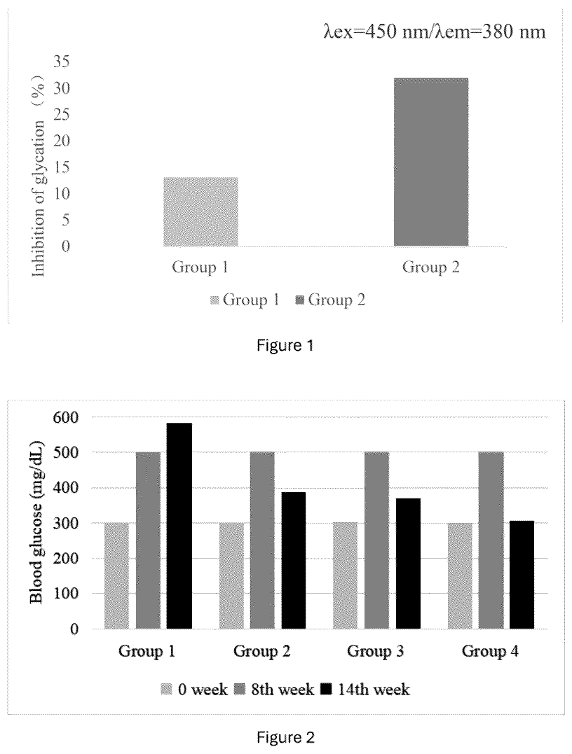

FIG. 1 is a graph of a percentage inhibition of fluorescence of the AGEs relative to the control. As shown in FIG. 1, EGT and EGT+DHB combination all have a fabulous anti-glycation activity. Especially, the DHB and EGT combination (Group 2) inhibited glycation remarkably up to about 32%.

### Example 2

Six-week-old, male, Kunming mice (n=40), weighed about 30±2 g, were obtained from Cavens Lab Animal (Changzhou, China) and housed in a controlled room (25±2° C., 60% relative humidity, 12 h light/dark cycle) with free access to water and standard laboratory diet. After an acclimation period (1 week), mice were randomly divided into four groups. Each mouse's back skin was chosen as the experimental observation area, a razor was used to shave off the back hair. 5% sodium sulfide solution was applied to the back to remove the hair.

All mice were then injected subcutaneously with same volume of D-galactose (1000 mg/kg), once a day for 8 weeks. During the entire experiment, the daily food intake and weight of each mouse were recorded every day, and the D-galactose injection dose were adjusted according to the weight change.

The four groups were non-supplement group (Con, Group 1); DHB supplement group (100 mg/kg, Group 2); EGT supplement group (100 mg/kg, Group 3); and EGT+DHB supplement group (100 mg/kg EGT+100 mg/kg DHB, Group 4). Mice in groups 2-4 were injected subcutaneously (s.c.) into their back skins with the corresponding concentration before each major meal once daily for 6 weeks.

All mice were sacrificed after 6 weeks at the end of the experiment. Skin tissue of each mouse was divided into three parts, the first part was stored at −80° C. until assays were performed, the second part was immediately collected, weighed and homogenized (4° C.; 3500 rpm/min for 15 min) for biochemical and histological analyses, and the third part was fixed in 10% formalin until further use.

### Measurement of Blood Glucose Level

When all mice were on fasting, approximately 10 μL of blood was collected by tail nick for 3 times, respectively at the beginning of the trial (0 week), after the D-galactose injection (8th week), and at the end of the trial (14th week). A glucometer was used to measure the glucose level.

FIG. 2 is a graph of blood glucose levels of four groups of mice at the beginning of the trial (0 week), after the D-galactose injection (8th week), and at the end of the trial (14th week). As shown in FIG. 2, AGEs were formed successfully by D-galactose. Fortunately, blood glucose levels were decreased significantly except group 1. At the end of the experiment, the blood glucose level was decreased by even 40% in the group 4. DHB and EGT combination thus is an advantageous substance for blood glucose control.

### Example 3

### Measurement of MDA

A commercial kit was used to confirm the content of MDA (an end product of lipid peroxidation) during the trial.

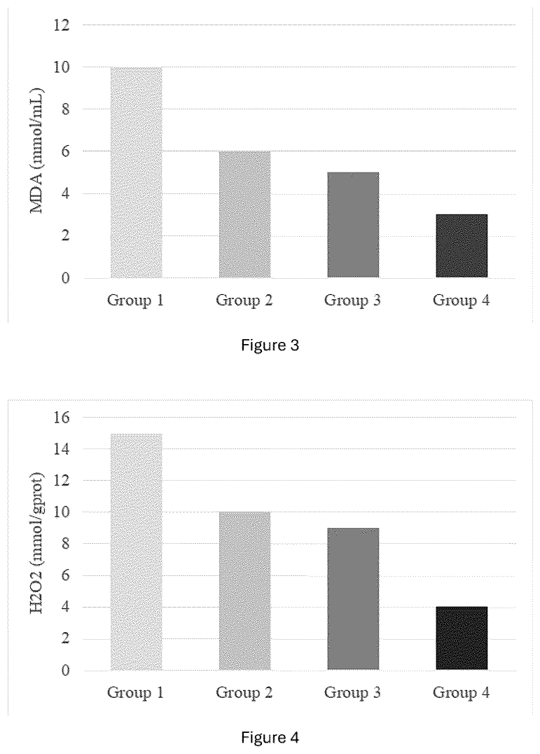

FIG. 3 is a graph of MDA content of four groups of mice at the end of the trial (14th week). As shown in FIG. 3, the content of MDA decreased to be normal in group 4, and was even 70% lower than that in non-supplement group. So, this combination could inhibit ROS-mediated lipid peroxidation significantly, such as reducing the MDA level.

Measurement of H₂O₂ and Oxidative Stress Levels

Oxidative stress is increased during aging. The changes of oxidoreductase activities should be paid high attention. Skins were lysed into homogenate using normal saline or RIPA tissue cracking agent. The levels of H₂O₂, SOD, CAT, GSH, and GSH-Px in the skin were detected with relative kits as reference. Measurement of the activities of four typical anti-oxidative enzymes, such as SOD, CAT, GSH, and GSH-Px could indicate whether the supplementation has the ROS-cleaning ability.

FIG. 4 is a graph of H₂O₂ content of four groups of mice at the end of the trial (14th week). As shown in FIG. 4, the H₂O₂ content was decreased about three times in group 4 as compared to the non-supplement group.

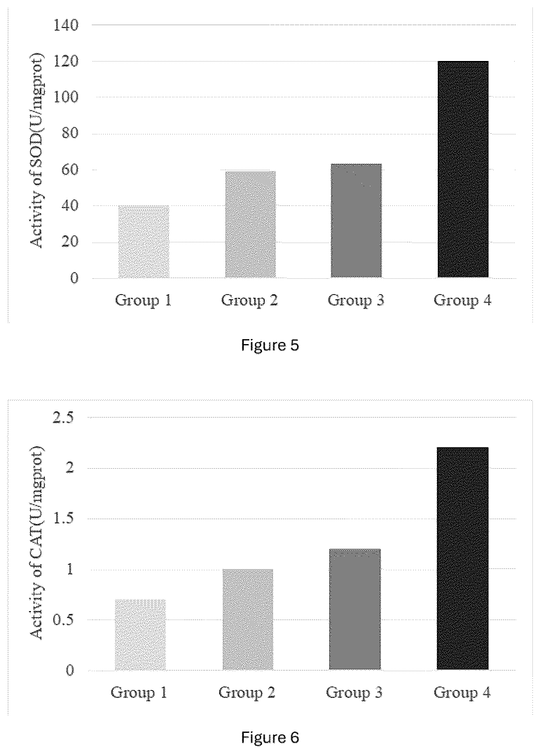

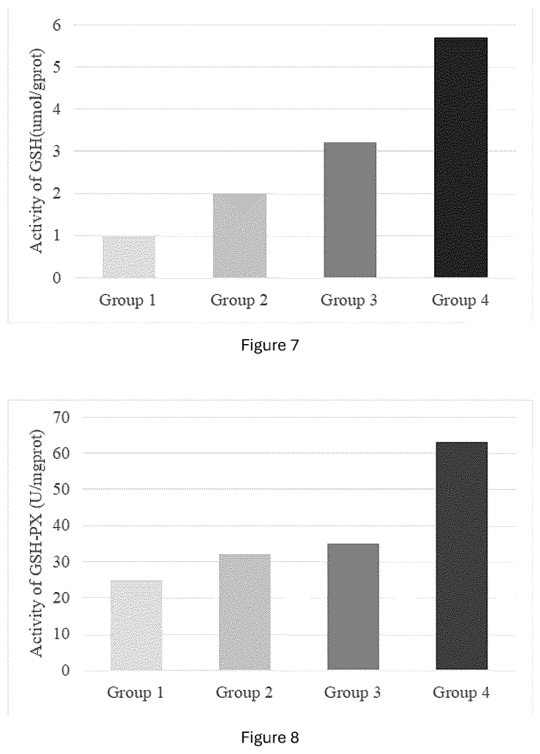

FIG. 5 is a graph of SOD activity of four groups of mice at the end of the trial (14th week). FIG. 6 is a graph of CAT activity of four groups of mice at the end of the trial (14th week). FIG. 7 is a graph of GSH activity of four groups of mice at the end of the trial (14th week). FIG. 8 is a graph of GSH-Px activity of four groups of mice at the end of the trial (14th week). As shown in FIGS. 5-8, the activities of four typical anti-oxidative enzymes were significantly improved after DHB or EGT or combination supplementation. Specifically, the SOD, CAT, GSH and GSH-Px activities in group 4 all increased markedly, compared to non-supplementation group. Thus, the supplementation can scavenge chronic ROS.

### Measurement of Expression of MMP-1

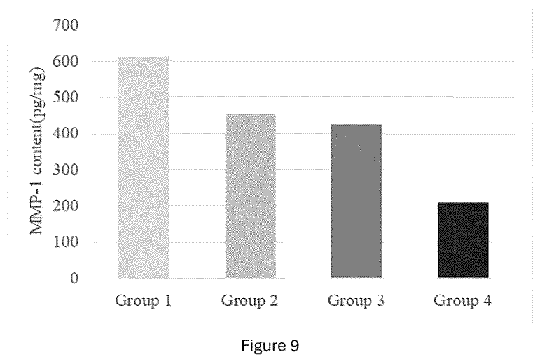

Quantitative polymerase chain reaction (QPCR) and western blot methods were used to measure the expression of MMP-1 in the skin. FIG. 9 is a graph of MMP-1 content of four groups of mice at the end of the trial (14th week). As shown in FIG. 9, the MMP-1 expression decreased significantly in group 4 (by about 3 times), compared to non-supplement group.

Therefore, the supplementation not only could reduce H₂O₂ production and MMP-1 expression during aging, but also exhibits superior anti-oxidation abilities and are effective at reducing oxidative stress in vivo.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

What is claimed is:

1. A method for reducing or inhibiting advanced glycation end products (AGEs) in a subject, comprising administrating to the subject a composition comprising: an effective amount of dihydroberberine or a pharmaceutically acceptable salt, acid, ester, analog or derivative thereof; and an effective amount of ergothioneine or a pharmaceutically acceptable salt, acid, ester, analog or derivative thereof.

2. The method of claim 1, wherein the method is used for anti-glycation.

3. The method of claim 1, wherein the method is used for mitigating or preventing aging.

4. The method of claim 3, wherein the aging is characterized by fine wrinkles, loss of elasticity, orange-peel or dull appearance of skin, reduced epidermal and dermal thickness, epidermal atrophy, decreased mitotic rate of basal keratinocytes, decreased proliferative capacity, cellular senescence, atrophy of dermal extracellular matrix, change of physiological properties of connective tissues.

5. The method of claim 1, wherein the reducing or inhibiting advanced glycation end products (AGEs) is achieved by controlling blood glucose, scavenging chronic ROS, reducing H₂O₂ and MDA productions and/or expression of MMP-1.

6. The method of claim 5, wherein the scavenging chronic ROS includes improving activities of anti-oxidative enzymes, such as SOD, CAT, GSH, and GSH-Px.

7. The method of claim 1, wherein the dihydroberberine or a pharmaceutically acceptable salt, acid, ester, analog or derivative thereof is administrated at a daily dose of 1-2000 mg, the ergothioneine or a pharmaceutically acceptable salt, acid, ester, analog or derivative thereof is administrated at a daily dose of 1-1500 mg.

8. The method of claim 1, wherein the ratio of the dihydroberberine or a pharmaceutically acceptable salt, acid, ester, analog or derivative thereof and the ergothioneine or a pharmaceutically acceptable salt, acid, ester, analog or derivative thereof ranges from 1:200 to 800:1.

9. The method of claim 1, wherein the subject is human or cattle or pet.

10. The method of claim 1, wherein the composition is prepared as a food, a drink, a supplement, a cosmetic product, or a pharmaceutical formulation.

11. The method of claim 1, wherein the administration is through various routes selected from oral administration, intravenous injection, intramuscular injection, intraperitoneal injection, topical application, or sublingual application.

12. The method of claim 1, wherein the composition is formulated in solutions, liquid suspensions, parenteral solutions, injections, tablets, pills, granules, powders, films, suppositories, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.
